(19) United States
(12) Patent Application Publication
(10) Pub. No.: US 2016/0157817 A1
(43) Pub. Date: Jun. 9, 2016

(54) APPARATUS AND METHOD FOR ULTRASOUND IMAGING OF AN EYE

(71) Applicant: NIDEK CO., LTD., Gamagori-shi (JP)

(72) Inventors: Cesare TANASSI, Susegana (IT); Raffaella BISSON, Padova (IT); Phil BUSCEMI, Mount Pleasant, SC (US)

(73) Assignee: NIDEK CO., LTD., Gamagori-shi (JP)

(21) Appl. No.: 14/962,549

(22) Filed: Dec. 8, 2015

(30) Foreign Application Priority Data
Dec. 9, 2014 (IT) CO2014A000039

(51) Int. Cl. A61B 8/00; A61B 8/10

(52) U.S. Cl. CPC A61B 8/4416; A61B 8/10; A61B 8/4411; A61B 8/4281; A61B 8/40; A61N 2007/0052

(57) ABSTRACT

There is provided an apparatus and method for ultrasound imaging a plurality of cross-sectional areas of a peripheral annular volume of an eye. The apparatus may include a mechanical device having a concave front surface designed to be located close to a front surface of a cornea of an eye, and a plurality of ultrasound emitting and receiving electronic devices associated with the mechanical device arranged in an annular manner so as to be distributed around the eye when the mechanical device is located close to the front surface of the cornea of the eye. The electronic devices may be directed toward a cross-sectional area of the peripheral annular volume of the eye. The mechanical device and electronic devices may be designed to be stationary with respect to the eye.

This application claims the benefit of Italian Application No. CO2014A000039, filed Dec. 9, 2014. The disclosure of the prior application is hereby incorporated by reference herein in its entirety.

### TECHNICAL FIELD

This application is directed to an apparatus and method for ultrasound imaging of an eye.

### BACKGROUND

Automatic ultrasound imaging of eyes for examination or therapeutic purposes is not widespread. For example, there are few devices available on the market today that provide ultrasound imaging of eyes for examination or therapeutic purposes.

One such known ultrasound device available on the market is known under the commercial name “ArcScan.”

Japanese publication JP S 62-106749A discloses a cornea thickness measuring ultrasonic probe. The thickness is measured by means of ultrasonic waves generated by a plurality of ultrasonic wave vibrators disposed on at least one meridian. The main measurement is in the center of the cornea. Other measurements are at different points of the cornea.

US publication 2012/089021A1 discloses a method and a system for three-dimensional imaging of biological structures, such as eyes. The ultrasonic scanning apparatus includes a unique ultrasonic array that slides back and forth along a linear translation rod during examination. The distance between the array and the eye continuously varies from, for example, a few centimeters to, for example, a few millimeters.

International publication WO 2015/070889A1 (published after the priority date of the present application) discloses a device and a method for performing thermal keratoplasty using high intensity focused ultrasounds. The device comprises a plurality of ultrasonic transducers for emitting ultrasound waves. The ultrasound waves of some transducers are focused on corresponding areas of the cornea in order to heat these areas and cause collagen shrinkage. Some transducers are capable of receiving ultrasound waves for ocular imaging of the areas of the cornea under treatment.

These and other conventional ultrasound devices include many moving parts that must move with high precision and accuracy with respect to the eye and, as a result, suffer many drawbacks. For example, the design of these devices is very complicated due to the requisite high precision and accuracy. Further, during operation of the device, it is inconvenient for the patient that has to dip his eye in a small tub full of liquid in order for the devices to function properly. These and other drawbacks are addressed by disclosed embodiments.

### SUMMARY

It is an object of disclosed embodiments to improve the technology of the eye ultrasound imaging and to provide a more simple equipment for ultrasound imaging a plurality of cross-sectional areas of a peripheral annular volume of an eye; preferably, such volume should include the iridocorneal part of the eye and also all the structures that are located behind the trabeculate and iris.

Disclosed embodiments leverage the flexibility of solid-state ultrasound emitting and receiving devices, in particular cMUTs. Due to their reduced dimensions, an array of devices may be arranged in, for example, an annular manner and positioned close to the eye scleral limbus in a stationary manner. In this way, cross-sectional images sections may be acquired quickly and easily.

In a first embodiment, there is provided an imaging apparatus for ultrasound imaging a plurality of cross-sectional areas of a peripheral annular volume of an eye. The apparatus comprises a first mechanical device having a front surface designed to be located proximal to a front surface of a cornea of the eye, and a plurality of ultrasound emitting and receiving electronic devices associated with the first mechanical device, the electronic devices being (i) arranged in an annular manner so as to be distributed around the eye when the mechanical device is located proximal to the front surface of the cornea of the eye, and (ii) directed toward the cross-sectional areas of the peripheral annular volume of the eye. The first mechanical device and the electronic devices are configured to be stationary with respect to the eye.

The apparatus may advantageously further comprise second mechanical device. The second mechanical device and the first mechanical device are fixed to each other (i.e., they do not move with respect to each other). The electronic devices are fixed to the first mechanical device (i.e., they do not move with respect to the first mechanical device).

According to some embodiments, the second mechanical device serves only as a disposable device.

According to some embodiments, the second mechanical device serves only as an optical device for the optical observation of an iridocorneal annular zone of the eye.

According to some embodiments, the second mechanical device serves both as a disposable device and as an optical device.

According to some embodiments, the first mechanical device serves also as an optical device for the optical observation of an iridocorneal annular zone of the eye.

In another embodiment, there is provided a method of ultrasound imaging a peripheral annular volume of an eye using a mechanical device having a concave front surface and a plurality of ultrasound emitting and receiving electronic devices associated with the mechanical device and arranged in an annular manner. The method comprises applying a viscous transparent coupling agent to at least one of a front surface of a cornea of the eye and the concave front surface of the first mechanical device, positioning the mechanical device so that the front surface of the mechanical device is proximal to the front surface of the eye and so that the electronic devices are distributed around the eye, and detecting a plurality of cross-sectional areas of the peripheral annular volume of the eye using the electronic devices. The electronic devices are directed toward the cross-sectional areas of the peripheral annular volume of the eye, and during the detecting of the plurality of cross-sectional areas, the mechanical device and the electronic devices are maintained stationary with respect to the eye.

According to some embodiments, the plurality of cross-sectional areas are detected contemporaneously or near-contemporaneously. The various detecting actions are typically independent from each other.

According to some embodiments, the method further comprises the step of disposing the device after the detecting step.

According to some embodiments, the device is transparent only to ultrasounds or only to (visible) light or to both ultrasounds and (visible) light.

According to some embodiments, both ultrasound imaging and optical observation are carried out.

Furthermore, treatment of tissues in the peripheral annular volume of the eye by radiating them with ultrasounds may be provided.

In another embodiment, there is provided a method of ultrasound imaging a peripheral annular volume of an eye using a first mechanical device having a front surface, a second mechanical device having a concave front surface and being transparent at least to ultrasounds, and a plurality of ultrasound emitting and receiving electronic devices associated with the first mechanical device and configured in an annular manner. The method comprises applying a viscous transparent coupling agent to at least one of a front surface of a cornea of the eye and the concave front surface of the second mechanical device, positioning the first mechanical device and the second mechanical device so that front surfaces of the first mechanical device and the second mechanical device are proximal to the front surface of the eye and so that the electronic devices are distributed around the eye, and detecting a plurality of cross-sectional areas of the peripheral annular volume of the eye using the plurality of ultrasound emitting and receiving electronic devices. The electronic devices are directed toward the cross-sectional areas of the peripheral annular volume of the eye, and during the detecting of the plurality of cross-sectional areas, the first mechanical device, the second mechanical device and the electronic devices are maintained stationary with respect to the eye.

### BRIEF DESCRIPTION OF THE DRAWINGS

The accompanying drawings, which are incorporated herein and constitute a part of the specification, illustrate disclosed embodiments and, together with the detailed description, explain these embodiments. In the drawings:

FIG. 1A shows a schematic cross-section view of a human eye;

FIG. 1B shows a schematic front view of a human eye together with a plurality of cross-sectional areas A and a corresponding plurality of ultrasound devices G;

FIG. 2A shows a schematic cross-section view of a human eye wherein two cross-sectional areas A are highlighted;

FIG. 2B shows an enlarged view of one of the cross-sectional areas A of FIG. 2A;

FIG. 3 shows a schematic cross-section view of an apparatus according to a first embodiment;

FIG. 4 shows a schematic cross-section view of an apparatus according to a second embodiment;

FIG. 5 shows a schematic tridimensional partial view of the apparatus of FIG. 4;

FIG. 6 shows a schematic cross-section view of an apparatus according to a third embodiment;

FIG. 7 shows a schematic tridimensional partial view of the apparatus of FIG. 6;

FIG. 8 shows a schematic cross-section view of an apparatus according to a fourth embodiment; and

FIG. 9 shows a schematic cross-section view of an apparatus according to a fifth embodiment.

### DETAILED DESCRIPTION

The following detailed description of disclosed embodiments refers to the accompanying drawings.

Although the detailed description focuses on exemplary embodiments, these embodiments are not intended to limit the scope of the disclosure. Various combinations, modifications, improvements and alternative presentations of disclosed embodiments are contemplated even if not explicitly disclosed herein.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

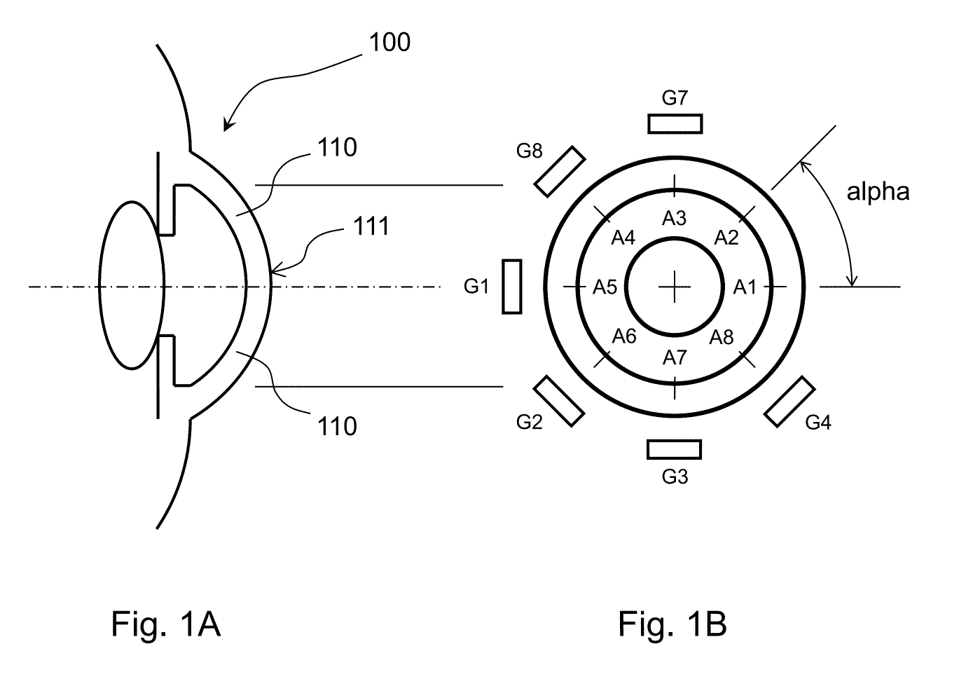

FIG. 1A shows a schematic cross-section view of a human eye 100. Reference 110 corresponds to the cornea and reference 111 corresponds to the front surface of the cornea.

FIG. 1B shows a schematic front view of a human eye 100 together with a plurality of cross-sectional areas A1, A2, A3, A4, A5, A6, A7, and A8 and a corresponding plurality of ultrasound emitting and receiving electronic devices G1, G2, G3, G4, G5, G6, G7, and G8. Device G1 (or a group of devices G1) is opposite to area A1 (with respect to the eye axis) and used for imaging area A1, device G2 (or a group of devices G2) is opposite to area A2 (with respect to the eye axis) and used for imaging area A2, and so on. The areas A and the devices G are regularly distributed around the eye and the angle between two adjacent areas A and corresponding devices G is defined by “alpha.” In embodiments, alpha is substantially equal or equal to 45°. While FIG. 1B shows eight areas and eight devices, disclosed embodiments are not particularly limited to this number of areas and devices such that a different number may be provided. Typically, the minimum number is four and the maximum number is sixteen.

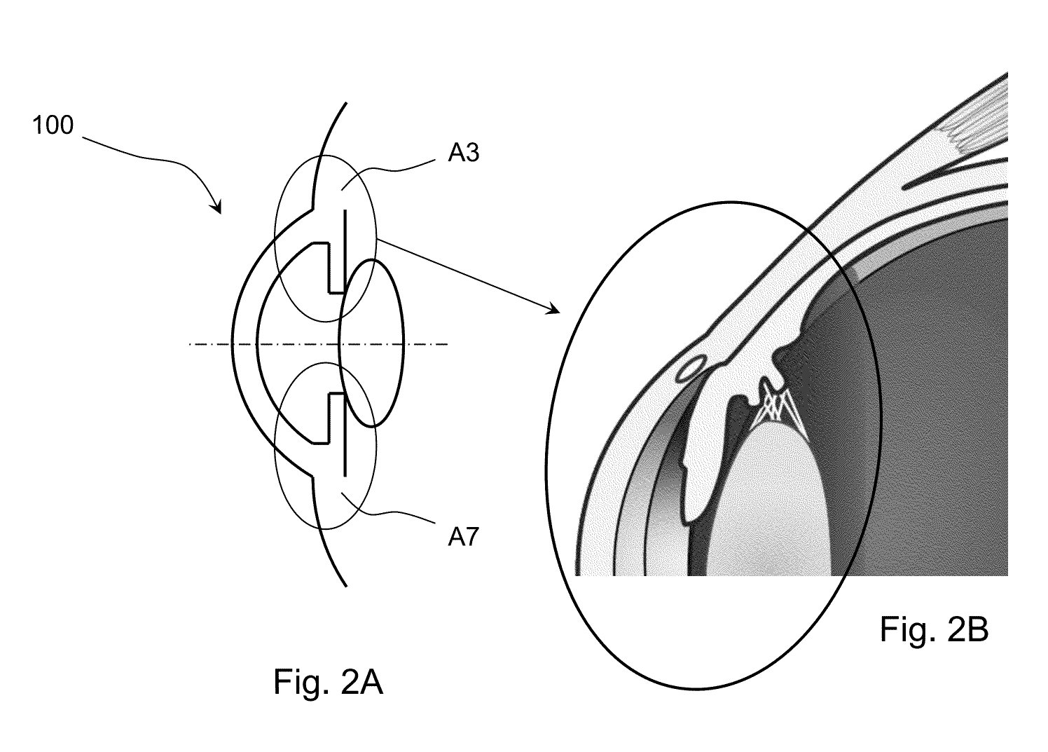

FIG. 2A shows a schematic cross-section view of the human eye 100 wherein two cross-sectional areas A3 and A7 are highlighted.

FIG. 2B shows an enlarged view of the cross-sectional area A3 of FIG. 2A. According to embodiments, the structures (including tissues) that should be imaged through ultrasound are preferably the cornea (and all its constituents), conjunctiva, angle of anterior chamber, trabecular meshwork, Schlemm's canal, scleral spur, iris, posterior chamber of the eye, ciliary body, Zinn's zonules, and crystalline lens. These structures are approximately contained in the ellipse of FIG. 2B.

Disclosed embodiments are directed to ultrasound imaging a peripheral annular volume of the anterior part of the eye, typically an internal volume. Preferably, an angle of at least 90° is covered through a single imaging process, more preferably the angle is at least 180°, or even more preferably, 360°.

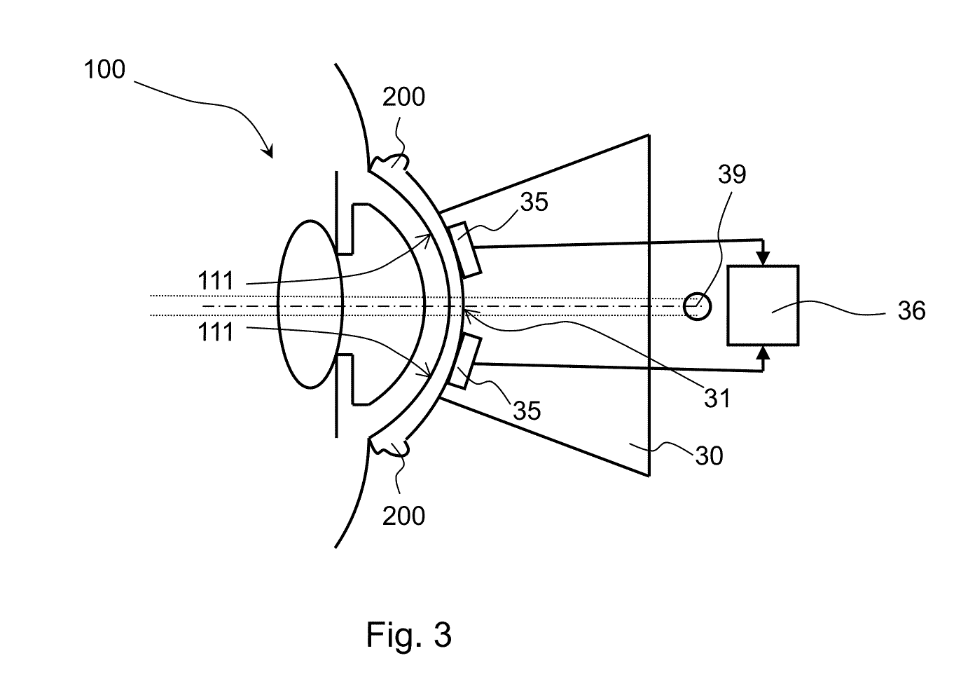

In embodiments, there is provided a method of using the mechanical device 30 shown in FIG. 3. The method may include: A) applying a viscous transparent coupling agent 200 between a front surface 111 of a cornea of the eye 100 and the front surface 31 of the mechanical device 30, B) positioning the mechanical device 30 so that the front surface 31 of the mechanical device 30 is close to the front surface 111 of the eye and so that the electronic devices 35 are distributed around the eye 100 when said mechanical device 30 is located close to the front surface 111 of the cornea 110 of the eye 100, each of said electronic devices 35 being directed toward a cross-sectional area (A1, A2, A3, A4, A5, A6, A7, and A8) of the peripheral annular volume of the eye 100. The method may further include C) detecting a plurality of cross-sectional areas (references A1, A2, A3, A4, A5, A6, A7, and A8) of the peripheral annular volume of the eye through the plurality of ultrasound emitting and receiving electronic devices 35 and G1, G2, G3, G4, G5, G6, G7, and G8. During step C, the mechanical device 30 is maintained stationary with respect to the eye and the electronic devices 35 and G1, G2, G3, G4, G5, G6, G7, and G8, are maintained stationary with respect to the eye.

It has been found that the distance between the mechanical device 30, specifically the front surface 31 of the mechanical device, and the cornea should be in the range of 0.5-2.5 mm, preferably in the range of 1.0-2.0 mm. Shorter distances and longer distances outside of these ranges have been found to be less effective and thus should be avoided. In particular, contact between the mechanical device and the eye should be avoided and a layer of a viscous transparent coupling agent should be between the mechanical device and the eye.

Suitable transparent (to both light and ultrasounds) coupling agents are currently available on the market and may include, for example, ophthalmic gels that are used for gonioscopic examinations.

Several devices allow for the implementation of disclosed methods, as discussed further below with respect to disclosed embodiments of the apparatus.

Preferably, each of the plurality of cross-sectional areas A1, A2, A3, A4, A5, A6, A7, and A8 is detected through a distinct group of electronic devices G1, G2, G3, G4, G5, G6, G7, and G8.

FIG. 3 illustrates a first embodiment.

With reference to FIG. 3, disclosed embodiments provide a mechanical device 30 having a front surface 31 and a plurality of ultrasound emitting and receiving electronic devices 35 corresponding to references G1, G2, G3, G4, G5, G6, G7, and G8 in FIG. 1B that are associated with the mechanical device 30 arranged in an annular manner. The electronic devices 35 may be integrated in the mechanical device 30 or mounted to the mechanical device so that the mechanical device is a support for the electronic devices. Alternatively, indirect support may be provided by the mechanic device.

According to this embodiment, the solid mechanical device 30 having a concave front surface 31 may be designed to be located close to a front surface 111 of a cornea an eye, in particular approximately matching the convex front surface 111 of the cornea of the eye. A plurality of ultrasound emitting and receiving electronic devices 35 may be fixed to the mechanical device 30. The mechanical device 30 may be designed to be stationary with respect to the eye (i.e. the apparatus does not comprise any actuator for device 30. The electronic devices 35 are designed to be stationary with respect to the eye (i.e. the apparatus does not comprise any actuator for devices 35.

The mechanical device 30 may have approximately the shape of a cylinder or a prism or a truncated cone or truncated pyramid, or is annular.

The electronic devices 35 may be cMUT devices.

Additionally, in embodiments, there may be an analog and/or digital signal processing circuitry 36 located close to the devices 35, for example, outside the mechanical device 30 but adjacent thereto.

There may also be a fixation target 39 for maintaining the eye in a fixed and predetermined position during ultrasound imaging. The so-called “fixation target” may be a small luminous device that attracts and maintains the attention of an eye. Centrally, the mechanical device 30 allows the propagation of the light beam of the fixation target 39 either because it is transparent to light or because it has a hole.

As each of the elements G1, G2, G3, G4, G5, G6, G7, and G8 may correspond to a group of ultrasound electronic devices, the group may be integrated in a microelectronic chip. Some analog and/or digital signal processing circuitry may be located in said chip and/or some analog and/or digital signal processing circuitry may be located close to said chip and/or some analog and/or digital signal processing circuitry may be located remote from said chip.

The plurality of cross-sectional areas (e.g., A1, A2, A3, A4, A5, A6, A7, and A8) detected by devices 35 cover the whole peripheral annular internal volume of the eye.

According to a variant of the embodiment of FIG. 3, the mechanical device may also be an optical device for the optical observation of an iridocorneal annular zone of the eye. In this case, the mechanical device 30 should allow propagation of light and the electronic devices 35 should not impede propagation of imaging light beams.

It is to be noted that, very advantageously, the electronic devices 35 may be arranged not only for imaging but also to treat tissues in the peripheral annular volume of the eye by radiating them with ultrasounds.

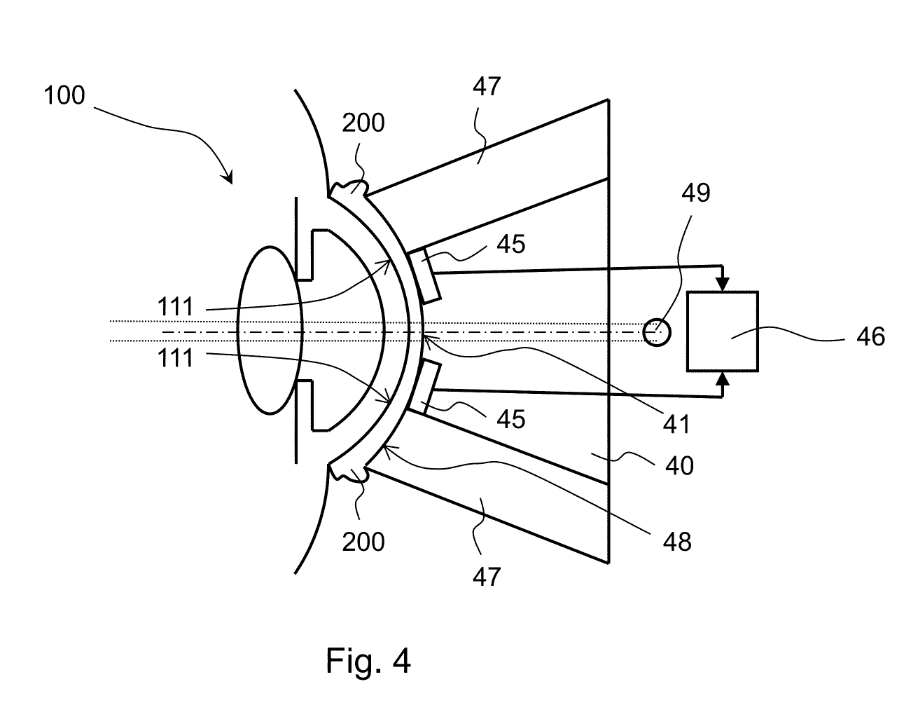

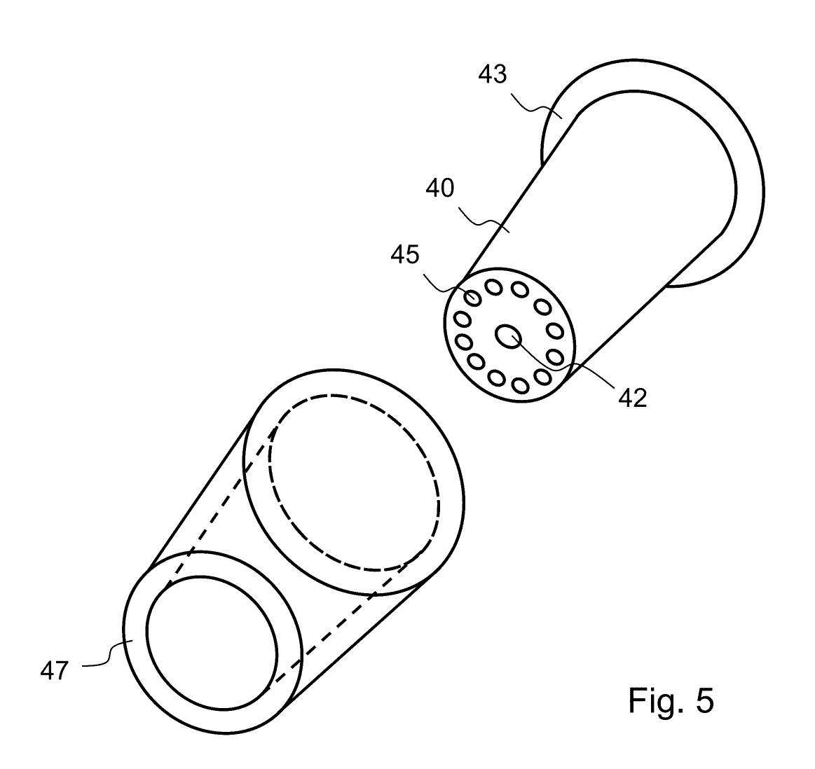

FIG. 4 and FIG. 5 illustrate a second embodiment.

This embodiment is similar to the embodiment of FIG. 3 but it further comprises another device 47. The other device 47 and the mechanical device 40 are fixed (integrated or mounted) to each other. In particular, the other device 47 is fixed (integrated or mounted) to the mechanical device 40 and the mechanical device 40 is inserted into a through hole of the other device 47 so that the other device 47 is located around the mechanical device 40.

FIG. 5 shows a hole 42 for the light beam of the fixation target 49 and a rim 43 for fixing (e.g., mounting) the devices together.

The other device 47 is an optical device for the optical observation of an iridocorneal annular zone of the eye.

The front surface 41 of device 40 and the front surface 48 of device 47 are substantially aligned on a common curved surface.

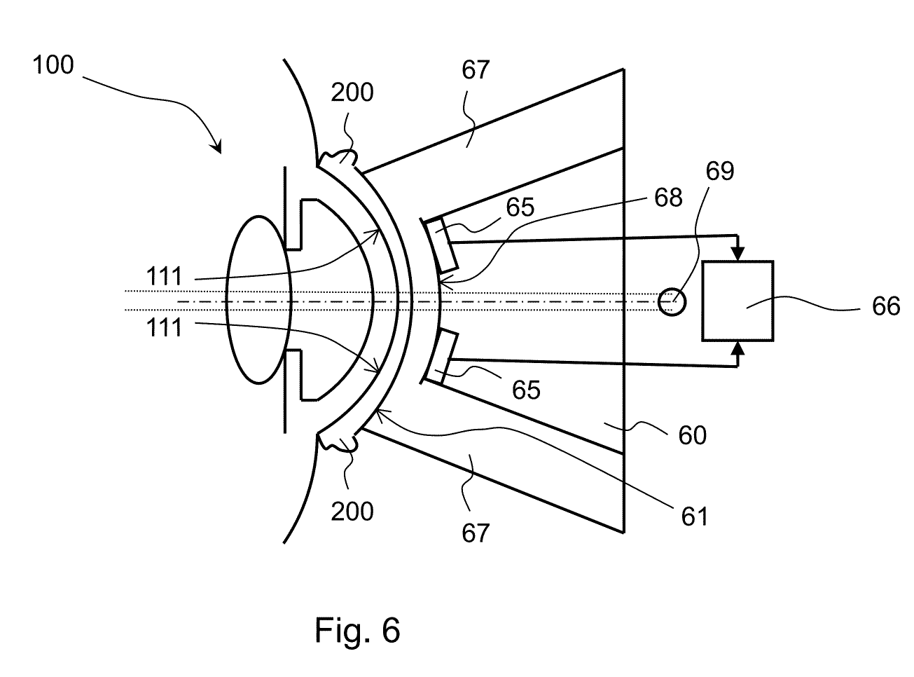

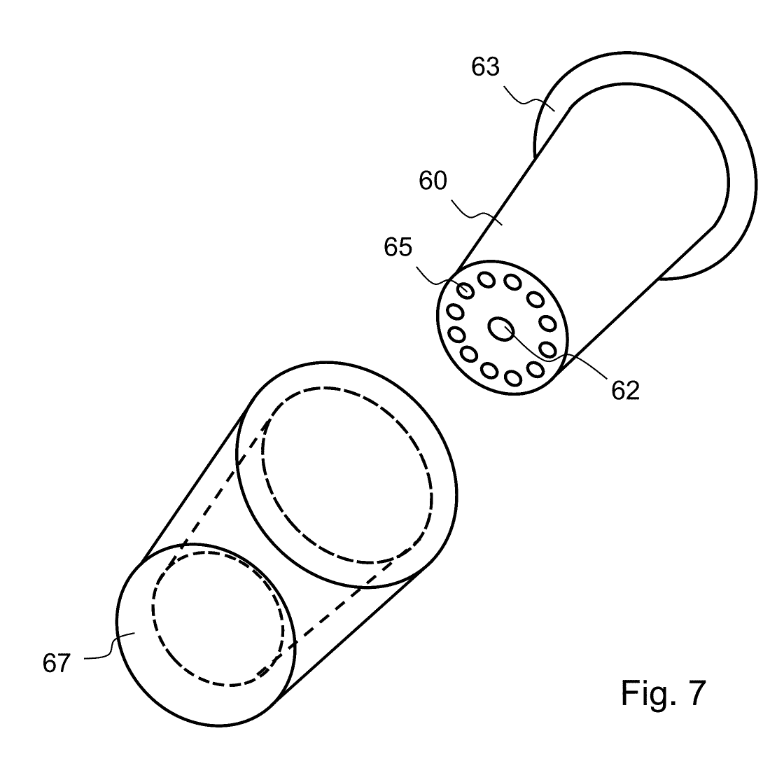

FIG. 6 and FIG. 7 illustrate a third embodiment.

This embodiment is similar to the embodiment of FIG. 4 and FIG. 5 but the hole of the other device 67 is a blind hole wherein the mechanical device 60 is located after having been inserted so that the other device 67 is located around the mechanical device 60.

According to this embodiment, the concave front surface 61 of the other device 67 is very close to the front surface 111 of the cornea of the eye and the front surface 68 of the mechanical device 60 is less close to the front surface 111 of the cornea of the eye being behind the front surface 61. Preferably, the concave front surface 61 substantially matches the convex front surface 111 of the cornea of the eye. Preferably, the front surface 68 is also concave.

According to this embodiment, at least the front portion of the other device 67 should be of a material transparent to both light and ultrasounds. A material that is sufficiently transparent to visible light and that allows good transmission of ultrasound waves may be, for example, PMMA [poly(methylmethacrylate)].

According to this embodiment, the other device 67 may be disposable and the mechanical device 60 may be permanent as it does not contact the eye, in particular the cornea, at all.

FIG. 7 shows a hole 62 for the light beam of the fixation target 69 and a rim 63 for fixing (e.g., mounting) the devices together.

The other device 67 is an optical device for the optical observation of an iridocorneal annular zone of the eye.

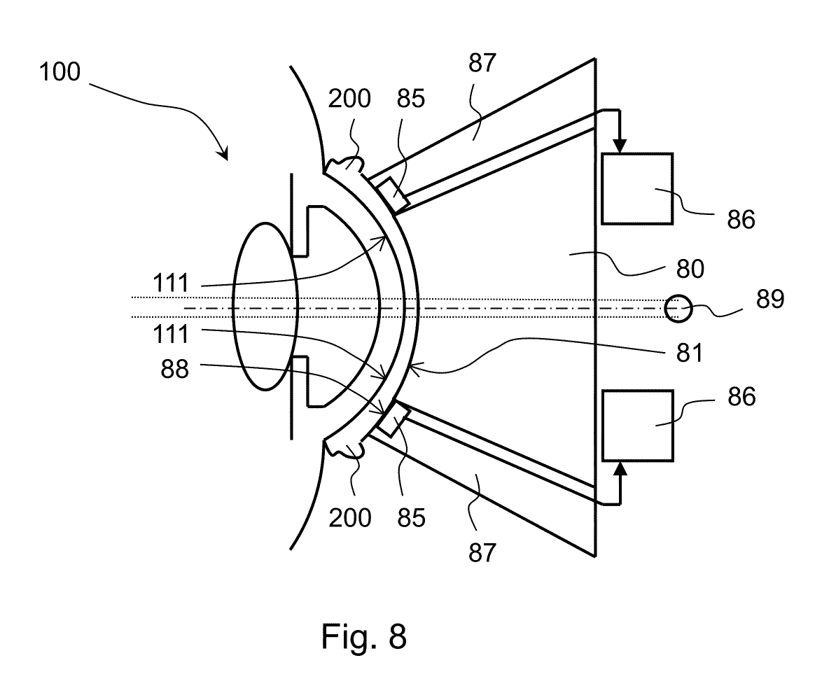

FIG. 8 illustrates a fourth embodiment.

The embodiment of FIG. 8 is similar to the embodiment of FIG. 4 but the mechanical device 87 is located around the other device 80, i.e., the optical device. The other device 87 and the mechanical device 80 are fixed (integrated or mounted) to each other. In particular, the mechanical device 87 is fixed (integrated or mounted) to the other device 80 and the other device 80 is inserted into a through hole of the mechanical device 87.

The front surface 81 of device 80 and the front surface 88 of device 87 are substantially aligned on a common curved surface.

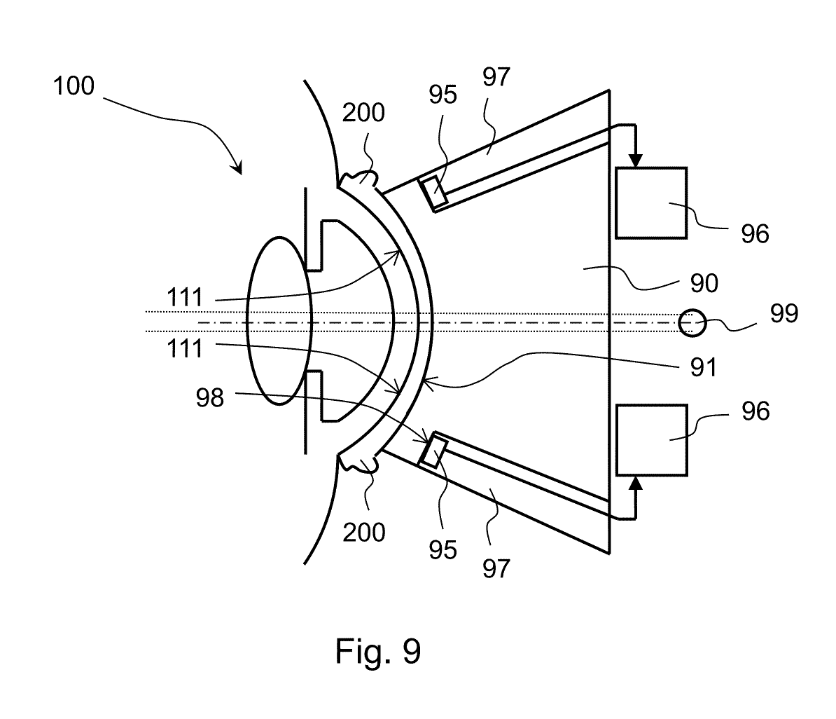

FIG. 9 illustrates a fifth embodiment.

The embodiment of FIG. 9 is similar to the embodiment of FIG. 6 but the mechanical device 97 is located around the other device 90, i.e., the optical device. In particular, device 97 is fixed (integrated or mounted) to the device 90 in a lateral recess of device 90.

According to this embodiment, the concave front surface 91 of the other device 90 is very close to the front surface 111 of the cornea of the eye and the front surface 98 of the mechanical device 97 is less close to the front surface 111 of the cornea of the eye being behind the front surface 91. Preferably, the concave front surface 91 substantially matches the convex front surface 111 of the cornea of the eye. Preferably, the front surface 98 is also concave.

According to this embodiment, at least the front portion of the other device 90 should be of a material transparent to both light and ultrasounds. A suitable material may be, for example, PMMA [poly(methylmethacrylate)].

According to this embodiment, the other device 90 may be disposable and the mechanical device 97 may be permanent as it does not contact the eye, in particular the cornea, at all.

In the embodiment of FIG. 3, the distance between the front surface of the mechanical device and the front surface of the cornea is typically in the range of 0.5-2.5 mm. A layer of a viscous transparent coupling agent should be between the eye and the mechanic device.

In the embodiment of FIG. 4, the distance between the front surface of the mechanical device and the front surface of the cornea is typically in the range of 0.5-2.5 mm, while the distance between the front surface of the other device and the front surface of the cornea is typically in the range of 0.5-2.5 mm. A layer of a viscous transparent coupling agent should be between the eye and the mechanical and other devices.

In the embodiment of FIG. 6, the distance between the front surface of the mechanical device and the front surface of the cornea is typically in the range of 5-20 mm, while the distance between the front surface of the other device and the front surface of the cornea is typically in the range of 0.5-2.5 mm. A layer of a viscous transparent coupling agent should be between the eye and the other device.

In the embodiment of FIG. 8, the distance between the front surface of the mechanical device and the front surface of the cornea is typically in the range of 0.5-2.5 mm, while the distance between the front surface of the other device and the front surface of the cornea is typically in the range of 0.5-2.5 mm. A layer of a viscous transparent coupling agent should be between the eye and the mechanical and other devices.

In the embodiment of FIG. 9, the distance between the front surface of the mechanical device and the front surface of the cornea is typically in the range of 5-20 mm, while the distance between the front surface of the other device and the front surface of the cornea is typically in the range of 0.5-2.5 mm. A layer of a viscous transparent coupling agent should be between the eye and the other device.

In the embodiments of FIG. 6 and FIG. 9, the front surface of the mechanical device is in contact with a rear surface of the other device.

In each of the disclosed embodiments, the electronic devices are at the front surface of the mechanical device.

For ease of reference, corresponding elements in the embodiments of FIG. 4 to FIG. 9 are as follows:
mechanical device (i.e. support device): 30, 40, 60, 87, 97
front surface of mechanic device: 31, 41, 68, 88, 98
other device (e.g. optical device): (not shown in FIG. 3), 47, 67, 80, 90
front surface of other device: (not shown in FIG. 3), 48, 61, 81, 91
electronic devices: 35, 45, 65, 85, 95
circuitry: 36, 46, 66, 86, 96
fixation target: 39, 49, 69, 89, 99

The apparatuses according to disclosed embodiments allow for implementation of disclosed methods.

These apparatuses allow for contemporaneous or near-contemporaneous detection of a plurality of cross-sectional areas (e.g., areas A1, A2, A3, A4, A5, A6, A7, and A8 in FIG. 1B) so that a single imaging process covers an angle of at least 90°, more preferably, at least 180°, and even more preferably, 360°. Typically, in case of non-contemporaneity, the maximum delay between two successive detections of two distinct groups of devices (e.g., the groups G1, G2, G3, G4, G5, G6, G7, and G8 in FIG. 1B) may be in the range of from 10 mS to 100 mS, and more preferably from 10 mS to 40 mS. The various detecting actions are typically independent from each other.

The apparatuses in the embodiments of FIG. 4, FIG. 6, FIG. 8, FIG. 9 and the mentioned variant of the device of FIG. 4, comprise both an ultrasound assembly and an optical assembly (embodied in two distinct devices or in a single device). Therefore, they provide not only ultrasound imaging but also optical observation of an iridocorneal annular zone of an eye. Advantageously, ultrasound imaging and optical observation are carried out contemporaneously or near-contemporaneously. Typically, in case of non-contemporaneity, the maximum delay between the two kinds of observations may be in the range of from 1 S or 10 S.

Alternatively, the other device in the embodiments of FIG. 4, FIG. 6, FIG. 8, and FIG. 9 may be only a support and/or cover for the mechanical device.

It will be appreciated that the above-disclosed features and functions, or alternatives thereof, may be desirably combined into different apparatuses or methods. Also, various alternatives, modifications, variations or improvements may be subsequently made by those skilled in the art, and are also intended to be encompassed by the following claims. As such, various changes may be made without departing from the spirit and scope of this disclosure as defined in the claims.

What is claimed is:

1. An imaging apparatus for ultrasound imaging a plurality of cross-sectional areas of a peripheral annular volume of an eye, the apparatus comprising:
a first mechanical device having a front surface designed to be located proximal to a front surface of a cornea of the eye; and
a plurality of ultrasound emitting and receiving electronic devices associated with the first mechanical device, the electronic devices being (i) arranged in an annular manner so as to be distributed around the eye when the mechanical device is located proximal to the front surface of the cornea of the eye, and (ii) directed toward the cross-sectional areas of the peripheral annular volume of the eye,
wherein the first mechanical device and the electronic devices are configured to be stationary with respect to the eye.

2. The imaging apparatus according to claim 1, further comprising a second mechanical device,
wherein the second mechanical device and the first mechanical device are fixed to each other, and
the electronic devices are fixed to the first mechanical device.

3. The imaging apparatus according to claim 2, wherein the second mechanical device is disposable and the first mechanical device is permanent.

4. The imaging apparatus according to claim 2, wherein the front surface of the first mechanical device is located behind a front surface of the second mechanical device.

5. The imaging apparatus according to claim 2, wherein the second mechanical device is an optical device for the optical observation of an iridocorneal annular zone of the eye.

6. The imaging apparatus according to claim 1, wherein the first mechanical device is an optical device for the optical observation of an iridocorneal annular zone of the eye.

7. The imaging apparatus according to claim 1, wherein the first mechanical device has the shape of a cylinder, a prism, a truncated cone, a truncated pyramid, or an annulus.

8. The imaging apparatus according to claim 1, wherein the electronic devices are cMUT devices.

9. The imaging apparatus according to claim 1, wherein each of the plurality of cross-sectional areas is detected through a corresponding group of electronic devices among the plurality of electronic devices.

10. The imaging apparatus according to claim 1, further comprising a fixation target configured to maintain the eye in a fixed and predetermined position during ultrasound imaging.

11. The imaging apparatus according to claim 1, wherein the electronic devices are configured to treat tissues in the peripheral annular volume of the eye by radiating the tissues with ultrasounds.

12. A method of ultrasound imaging a peripheral annular volume of an eye using a mechanical device having a concave front surface and a plurality of ultrasound emitting and receiving electronic devices associated with the mechanical device and arranged in an annular manner, the method comprising:
applying a viscous transparent coupling agent to at least one of a front surface of a cornea of the eye and the concave front surface of the first mechanical device;
positioning the mechanical device so that the front surface of the mechanical device is proximal to the front surface of the eye and so that the electronic devices are distributed around the eye; and
detecting a plurality of cross-sectional areas of the peripheral annular volume of the eye using the electronic devices,
wherein the electronic devices are directed toward the cross-sectional areas of the peripheral annular volume of the eye, and
during the detecting of the plurality of cross-sectional areas, the mechanical device and the electronic devices are maintained stationary with respect to the eye.

13. The method of ultrasound imaging according to claim 12, wherein the plurality of cross-sectional areas are detected contemporaneously or near-contemporaneously.

14. The method of ultrasound imaging according to claim 12, wherein the mechanical device is transparent to light and is configured to be used as an optical device for optical observation of an iridocorneal annular zone of the eye, and
ultrasound imaging and optical observation are carried out contemporaneously or near-contemporaneously.

15. A method of ultrasound imaging a peripheral annular volume of an eye using a first mechanical device having a front surface, a second mechanical device having a concave front surface and being transparent at least to ultrasounds, and a plurality of ultrasound emitting and receiving electronic devices associated with the first mechanical device and configured in an annular manner, the method comprising:
applying a viscous transparent coupling agent to at least one of a front surface of a cornea of the eye and the concave front surface of the second mechanical device;
positioning the first mechanical device and the second mechanical device so that front surfaces of the first mechanical device and the second mechanical device are proximal to the front surface of the eye and so that the electronic devices are distributed around the eye; and
detecting a plurality of cross-sectional areas of the peripheral annular volume of the eye using the plurality of ultrasound emitting and receiving electronic devices,
wherein the electronic devices are directed toward the cross-sectional areas of the peripheral annular volume of the eye, and
during the detecting of the plurality of cross-sectional areas, the first mechanical device, the second mechanical device and the electronic devices are maintained stationary with respect to the eye.

16. The method of ultrasound imaging according to claim 15, wherein the plurality of cross-sectional areas are detected contemporaneously or near-contemporaneously.

17. The method of ultrasound imaging according to claim 15, further comprising disposing the second mechanical device after the detecting of the plurality of cross-sectional areas.

18. The method of ultrasound imaging according to claim 15, wherein the second mechanical device is transparent to light and configured to be used for optical observation of an iridocorneal annular zone of the eye, and
ultrasound imaging and optical observation are carried out contemporaneously or near-contemporaneously.
